(19) United States
(12) Patent Application Publication
(10) Pub. No.: US 2017/0189539 A1
(43) Pub. Date: Jul. 6, 2017

(54) COMPOSITION HAVING IMPROVED SPRAYING CHARACTERISTICS COMPRISING VEGETABLE OILS AND TRIGLYCERIDES AND/OR MINERAL OILS

(71) Applicant: PARAGON NORDIC AB, Vallentuna (SE)

(72) Inventors: Malin BURSTEDT, Vallentuna (SE); Michael WALLIN, Enebyberg (SE); Mats SILVANDER, Upplands Väsby (SE)

(21) Appl. No.: 15/309,952

(22) PCT Filed: May 12, 2015
(86) PCT No.: PCT/SE2015/050525

§ 371 Date: Nov. 9, 2016

(30) Foreign Application Priority Data
May 13, 2014 (SE) 1450558-0

(51) Int. Cl. A61K 47/44; A61M 15/08; A61K 47/14; A61K 9/00; A61K 9/12

(52) U.S. Cl. CPC A61K 47/44; A61K 9/0043; A61K 9/12; A61K 47/14; A61M 15/08

(57) ABSTRACT

A composition with improved spray characteristics consists of i) a lipophilic component comprising 40-60% by weight of a medium to high viscosity vegetable oil or oil mixture and 40-60% by weight of low viscous triglyceride(s) and/or mineral oil(s), and optionally ii) 0-5% by weight of surfactant(s), iii) 0-5% by weight of pharmaceutically active agent(s), and iv) 0-20% by weight of propellant(s).

### TECHNICAL FIELD

The present embodiments generally relate to compositions, such as intranasal compositions, and in particular compositions having improved spraying characteristics.

### BACKGROUND

There are several prior art oil-based nasal sprays available on the market. Many of these nasal sprays use sesame oil as main lipophilic component due to its publically documented beneficial properties. Examples of such prior art nasal sprays based on sesame oil are disclosed in WO 2005/048952.

As a nasal spray, sesame oil acts as a barrier to prevent the loss of moisture from the nasal lining (mucous membrane), thereby restoring the natural moisture levels to nasal passages. Sesame oil also acts as a softener which induces scabs, clots, hardened mucus, etc. to separate, thereby promoting their manageability. The anti-inflammatory qualities of various naturally occurring minor components of sesame oil have been noted. Individuals suffering from the discomforting effects of colds, sinusitis and associated nasal inflammation (characterized by dry, irritated, crusty nasal passages, often accompanied by bleeding or bloody discharges), have traditionally sought relief from such discomforts through the use of isotonic saline solution. However, in a randomized crossover clinical study designed to compare the quality of relief from the symptoms of dry mucosa, medical researchers tested isotonic saline solution against pure sesame oil and found that sesame oil was significantly more effective than saline solution at relieving the associated dryness, crustiness and stuffiness that afflict individuals suffering from dry mucous membrane (Johnsen et al., Arch Otolaryngol Head Neck Surg, 2001, 127, pages 1353-1356.)

However, nasal sprays based on sesame oil and other medium to high viscosity vegetable oils or oil mixtures have a less than optimal spraying pattern and characteristics. This is in part dependent on the high viscosity of the used oils and sesame oil in particular. Sesame oil has a fairly high viscosity, 60 centipoise (cP) at room temperature (20°).

There is therefore a need for compositions, such as intranasal compositions, based on a medium to high viscosity vegetable oil or oil mixture, such as sesame oil, that has improved spraying characteristics.

### SUMMARY

It is a general objective to provide a composition having improved spraying characteristics.

This and other objectives are met by embodiments as disclosed herein.

An aspect of the embodiments relates to a composition consisting of a lipophilic component comprising a medium to high viscosity vegetable oil or oil mixture at 40-60% by weight of the lipophilic component. The medium to high viscosity vegetable oil or oil mixture has a viscosity above 50 centipose (cP) at room temperature (20° C.). The medium to high viscosity vegetable oil or oil mixture comprises mono- and/or polyunsaturated fats at at least 40% by weight of the medium to high viscosity vegetable oil or oil mixture. The lipophilic component also comprises a low viscous triglyceride or a mixture of low viscous triglycerides and/or a low viscous mineral oil or a mixture of low viscous mineral oils at 40-60% by weight of the lipophilic component. The composition optionally also consists of a surfactant or a mixture of surfactants at 0-5% by weight of the composition, a pharmaceutically active agent or a mixture of pharmaceutically active agents at 0-5% by weight of the composition, and a propellant or a mixture of propellants at 0-20% by weight of the composition.

Another aspect of the embodiments relates to a composition comprising a lipophilic component at 90-99.9% by weight of the composition. The lipophilic component comprises a medium to high viscosity vegetable oil or oil mixture at 40-60% by weight of the lipophilic component. The medium to high viscosity vegetable oil or oil mixture has a viscosity above 50 cP at room temperature (20° C.). The medium to high viscosity vegetable oil or oil mixture comprises mono- and/or polyunsaturated fats at at least 40% by weight of the medium to high viscosity vegetable oil or oil mixture. The lipophilic component also comprises a mixture of capric triglyceride and caprylic triglyceride at 40-60% by weight of the lipophilic component. The composition also comprises a surfactant or a mixture of surfactants at 0.1-5% by weight of the composition.

A further aspect of the embodiments relates to a composition according to above for use in treating, preventing and/or relieving symptoms of a nasal medical condition in a human subject. The nasal medical condition is selected from a group consisting of dry nasal mucous membrane, irritated nasal mucous membrane, sinus infection, rhinosinusitis, allergic rhinitis, and nasal swelling.

A related aspect of the embodiments defines a method of treating, preventing and/or relieving a nasal medical condition selected from a group consisting of dry nasal mucous membrane, irritated nasal mucous membrane, sinus infection, rhinosinusitis, allergic rhinitis, and nasal swelling in a human subject. The method comprises intranasally administering a composition according to above to the human subject.

Yet another aspect of the embodiments relates to a composition comprising a lipophilic component at 80-100% by weight of the composition for use in treating, preventing and/or relieving symptoms of a nasal medical condition in a human subject. The lipophilic component comprises a medium to high viscosity vegetable oil or oil mixture at 40-60% by weight of the lipophilic component. The medium to high viscosity vegetable oil or oil mixture has a viscosity above 50 cP at room temperature (20° C.). The medium to high viscosity vegetable oil or oil mixture comprises mono- and/or polyunsaturated fats at at least 40% by weight of the medium to high viscosity vegetable oil or oil mixture. The lipophilic component also comprises a mixture of capric triglyceride and caprylic triglyceride at 40-60% by weight of the lipophilic component. The nasal medical condition is selected from a group consisting of dry nasal mucous membrane, irritated nasal mucous membrane, sinus infection, rhinosinusitis, allergic rhinitis, and nasal swelling.

A related aspect of the embodiments defines a method of treating, preventing and/or relieving a nasal medical condition selected from a group consisting of dry nasal mucous membrane, irritated nasal mucous membrane, sinus infection, rhinosinusitis, allergic rhinitis, and nasal swelling in a human subject. The method comprises intranasally administering a composition comprising a lipophilic component at 80-100% by weight of the composition to the human subject. The lipophilic component comprises a medium to high viscosity vegetable oil or oil mixture at 40-60% by weight of the lipophilic component. The medium to high viscosity vegetable oil or oil mixture has a viscosity above 50 cP at room temperature (20° C.). The medium to high viscosity vegetable oil or oil mixture comprises mono- and/or polyunsaturated fats at at least 40% by weight of the medium to high viscosity vegetable oil or oil mixture. The lipophilic component also comprises a mixture of capric triglyceride and caprylic triglyceride at 40-60% by weight of the lipophilic component.

A further aspect of the embodiments relates to a nasal spray delivery device comprising a nozzle configured to be inserted into a nostril of a human subject and a composition container in fluid connection with the nozzle. The composition container comprises a composition according to above.

Yet another aspect of the embodiments relates to use of a low viscous triglyceride or a mixture of low viscous triglycerides and/or a low viscous mineral oil or a mixture of low viscous mineral oils as an additive to a composition comprising a medium to high viscosity vegetable oil or oil mixture to improve spray characteristics during administration of the composition as a spray or aerosol. The medium to high viscosity vegetable oil or oil mixture has a viscosity above 50 cP at room temperature (20° C.). The medium to high viscosity vegetable oil or oil mixture comprises mono- and/or polyunsaturated fats at at least 40% by weight of the medium to high viscosity vegetable oil or oil mixture.

### BRIEF DESCRIPTION OF THE DRAWINGS

The embodiments, together with further objects and advantages thereof, may best be understood by making reference to the following description taken together with the accompanying drawings, in which:

FIG. 1 is a diagram illustrating a comparison of spray pattern diameters of a reference nasal spray and a first test nasal spray (n=4);

FIG. 2 illustrates spray patterns from the reference nasal spray (FIG. 2A) and the first test nasal spray (FIG. 2B);

FIG. 3 is a diagram illustrating a comparison of vertical flow distances of the reference nasal spray and the first test nasal spray (n=4);

FIG. 4 illustrates vertical flow of the reference nasal spray (FIG. 4A) and the first test nasal spray (FIG. 4B) on intact skin;

FIG. 5 illustrate spray patterns into paper cones for the reference nasal spray (FIG. 5A) and the first test nasal spray (FIG. 5B);

FIG. 6 illustrates spray patterns from the reference nasal spray (FIG. 6A) and a second test nasal spray (FIG. 6B);

FIG. 7 is a diagram illustrating a comparison of vertical flow distances of the reference nasal spray and the second test nasal sprays (n=4);

FIG. 8 illustrates vertical flow of the reference nasal spray (FIG. 8A) and the second test nasal spray (FIG. 8B) on intact skin;

FIG. 9 illustrate spray patterns into paper cones for the reference nasal spray (FIG. 9A) and the second test nasal spray (FIG. 9B); and

FIG. 10 illustrates results for spray pattern tests performed with test nasal sprays according to the embodiments and reference nasal sprays.

### DETAILED DESCRIPTION

The present embodiments generally relate to compositions, such as intranasal compositions, and in particular compositions having improved spraying characteristics.

Compositions, such as intranasal compositions, such as intranasal sprays and aerosols, comprising medium to high viscosity vegetable oil or oil mixture, such as sesame oil, and in particular consisting to a major part of sesame oil, have shortcomings due to the comparatively high viscosity of the oil. For instance, sesame oil typically has a viscosity of about 60 centipoise (cP) at room temperature (20° C.). The high viscosity results in non-optimal characteristics and properties of the composition including, for instance, low adsorption onto skin and mucous membrane and instead forming a running product on the skin or membrane surface. Furthermore, the spray pattern of compositions consisting of pure medium to high viscosity vegetable oil or oil mixture, such as sesame oil or substantially consisting of only sesame oil, will be a central jet of oil forming running droplets on the target surface.

The present embodiments solve at least some of the above mentioned shortcomings of compositions with medium to high viscosity vegetable oil or oil mixture. Thus, as compared to substantially pure oils, compositions of the embodiments have high degree of adsorption onto skin and mucous membrane and result in a spray mist onto a target surface.

The present embodiments use a lipophilic component not only comprising medium to high viscosity vegetable oil or oil mixture but also comprising low viscous triglyceride(s) and/or low viscous mineral oil(s). The addition of such low viscous triglyceride(s) and/or mineral oil(s) to the medium to high vegetable oil significantly lowers the viscosity of the resulting composition at room temperature. The resulting reduction in viscosity of the composition is believed to be at least partly the basis for the improvements seen by the compositions of the embodiments over prior art compositions as shown in the experiment section.

As aspect of the embodiments relates to a composition consisting of a liphophilic component, a surfactant or a mixture of surfactants at 0-5% by weight of the composition, a pharmaceutically active agent or a mixture of pharmaceutically active agents at 0-5% by weight of the composition and a propellant or a mixture of propellants at 0-20% by weight of the composition. The lipophilic component comprises a medium to high viscosity vegetable oil or oil mixture at 40-60% by weight of the lipophilic component and a low viscous triglyceride or a mixture of low viscous triglycerides and/or a low viscous mineral oil or a mixture of low viscous mineral oils at 40-60% by weight of the lipophilic component. The medium to high viscosity oil or oil mixture has a viscosity above 50 cP at room temperature (20° C.) and comprises mono- and/or polyunsaturated fats at at least 40% by weight of the medium to high viscosity vegetable oil or oil mixture.

Thus, in this aspect of the embodiments the composition consists of at least a lipophilic component. The composition may optionally also include surfactant(s), pharmaceutically active agent(s) and/or propellant(s).

In an embodiment, the composition consists of the lipophilic component comprising a medium to high viscosity vegetable oil or oil mixture at 40-60% by weight of the lipophilic component (and thereby of the composition) and a low viscous triglyceride or a mixture of low viscous triglycerides and/or a low viscous mineral oil or a mixture of low viscous mineral oils at 40-60% by weight of the lipophilic component (and thereby of the composition). In this embodiment, the composition thereby solely consists of the lipophilic component and does not comprise any additional components or ingredients.

In another embodiment, the composition consists of the lipophilic component and a surfactant or a mixture of surfactants up to an amount of no more than 5% by the weight of the composition.

In a further embodiment, the composition consists of the lipophilic component and a pharmaceutically active agent or a mixture of pharmaceutically active agents up to an amount of no more than 5% by weight of the composition.

In yet another embodiment, the composition consists of the lipophilic component and a propellant or a mixture of propellants up to an amount of no more than 20% by weight of the composition.

In still another embodiment, the composition consists of the lipophilic component, a surfactant or a mixture of surfactants up to an amount of no more than 5% by the weight of the composition, and a pharmaceutically active agent or a mixture of pharmaceutically active agents up to an amount of no more than 5% by weight of the composition.

In a further embodiment, the composition consists of the lipophilic component, a surfactant or a mixture of surfactants up to an amount of no more than 5% by the weight of the composition, and a propellant or a mixture of propellants up to an amount of no more than 20% by weight of the composition.

In another embodiment, the composition consists of the lipophilic component, a pharmaceutically active agent or a mixture of pharmaceutically active agents up to an amount of no more than 5% by weight of the composition, and a propellant or a mixture of propellants up to an amount of no more than 20% by weight of the composition.

In a further embodiment, the composition consists of the lipophilic component, a surfactant or a mixture of surfactants up to an amount of no more than 5% by the weight of the composition, a pharmaceutically active agent or a mixture of pharmaceutically active agents up to an amount of no more than 5% by weight of the composition, and a propellant or a mixture of propellants up to an amount of no more than 20% by weight of the composition.

In a particular embodiment, the composition consists of the lipophilic component at 90-100% by weight of the composition, the surfactant or the mixture of surfactants at 0-5% by weight of the composition, the pharmaceutically active agent or the mixture of pharmaceutically active agents at 0-5% by weight of the composition, and the propellant or the mixture of propellants at 0-10% by weight of the composition.

The lipophilic component comprises a medium to high viscosity vegetable oil or oil mixture at 40-60% by weight of the lipophilic component. According to the embodiments, the medium to high viscosity vegetable oil or oil mixture has a viscosity above 50 cP at room temperature (20°). In a particular embodiment, the medium to high viscosity vegetable oil or oil mixture has a viscosity within an interval of 50 to 100 cP at room temperature (20°), preferably within an interval of 60 to 90 cP at room temperature) (20°).

The medium to high viscosity vegetable oil or oil mixture comprises mono- and/or polyunsaturated fats at at least 40% by weight of the medium to high viscosity vegetable oil or oil mixture. Hence, the medium to high viscosity vegetable oil or oil mixture comprises a total amount of unsaturated fats of at least 40% by weight of the medium to high viscosity vegetable oil or oil mixture. The unsaturated fats of the medium to high viscosity vegetable oil or oil mixture could be monounsaturated fats, polyunsatured fats or a mixture of mono- and polyunsatured fats.

In a particular embodiment, the medium to high viscosity vegetable oil or oil mixture comprises the mono- and/or polyunsatured fats at at least 50% by weight, preferably at at least 60% by weight, of the medium to high viscosity vegetable oil or oil mixture.

In an embodiment, the mono- and/or polyunsaturated fats are selected from a group consisting of palmitoleic acid ((9Z)-hexadec-9-enoic acid), oleic acid ((9Z)-octadec-9-enoic acid), linoleic acid ((9Z,12Z)-9,12-octadecadienoic acid), linolenic acid ((9Z,12Z,15Z)-9,12,15-octadecatrienoic acid, α-linolenic acid and/or all-cis-6,9,12-octadecatrienoic acid, χ-linolenic acid), eicosenoic acid ((9Z)-9-Icosenoic acid, gadoleic acid or 9-eicosenoic acid; (Z)-eicos-11-enoic acid, gondoic acid or 11-eicosenoic acid; and/or (13Z)-13-eicosenoic acid, paullinic acid or 13-eicosenoic acid).

In an embodiment, the medium to high viscosity vegetable oil or oil mixture comprises at least one antioxidant. In a particular embodiment, the at least one antioxidant is selected from a group consisting of vitamin E, vitamin K and carotenoids.

Vitamin E may be in the form of a tocopherol, a mixture of tocopherols, a tocotrienol, a mixture of tocotrienols, or a mixture of at least one tocopherol and at least one tocotienol. Examples of tocopherols include α-tocopherol, β-tocopherol, χ-tocopherol, δ-tocopherol and ε-tocopherol. Examples of tocotrienols include α-tocotrienol, β-tocotrienol, χ-tocotrienol, δ-tocotrienol and ε-tocotrienol.

Vitamin K comprises 2-methyl-1,4-naphthoquinone (3-) derivatives and includes the two natural vitamins vitamin K₁ (also known as phylloquinone, phytomenadione, or phytonadione) and vitamin K₂ (also known as menaquinones or MK-n, wherein M stands for menaquinone, K stands for vitamin K, and n represents the number of isoprenoid side chain residues, for instance n=4, 7, 8 or 9).

Carotenoids are organic pigments that are found in the chloroplasts and chromoplasts of plants and some other photosynthetic organisms, including some bacteria and some fungi. Carotenoids are generally classified as xanthophylls or carotenes. Non-limiting examples of suitable carotenoids include β-carotene, α-carotene, and β-cryptoxanthin. Further non-limiting examples of carotenoids include lycopersene, phytofluene, hexahydrolycopene, torulene, α-zeacarotene, alloxanthin, cynthiaxanthin, pectenoxanthin, cryptomonaxanthin, crustaxanthin, gazaniaxanthin, OH-chlorobactene, loroxanthin, lutein, lycoxanthin, rhodopsin, rhodopinol, saproxanthin, zeaxanthin, oscillaxanthin, phleixanthophyll, rhodovibrin, spheroidene, diadinoxanthin, luteoxanthin, mutatoxanthin, citroxanthin, zeaxanthin furanoxide, neochrome, foliachrome, trollichrome, vaucheriaxanthin, rhodopinal, warmingone, torularhodinaldehyde, torularhodin, torularhodin methyl ester, astacene, astaxanthin, canthaxanthin, capsanthin, capsorubin, cryptocapsin, 2,2′-diketospirilloxanthin, flexixanthin, 3-OH-canthaxanthin, hydroxyspheriodenone, okenone, pectenolone, phoeniconone, phoenicopterone, rubixanthone, siphonaxanthin, astacein, fucoxanthin, isofucoxanthin, physalien, zeaxanthin, siphonein, apocarotenoids, apo-2-lycopenal, apo-6′-lycopenal, azafrinaldehyde, bixin, citranaxanthin, crocetin, crocetinsemialdehyde, crocin, hopkinsiaxanthin, methyl apo-6′-lycopenoate, paracentrone, sintaxanthin, actinioerythrin, β-Carotenone, peridinin, pyrrhoxanthininol, semi-α-carotenone, semi-β-carotenone, triphasiaxanthin, eschscholtzxanthin, eschscholtzxanthone, rhodoxanthin, tangeraxanthin, nonaprenoxanthin, decaprenoxanthin, 2-[4-hydroxy-3-(hydroxymethyl)-2-butenyl]-2′-(3-methyl-2-butenyl)-b,b-carotene, 2′-(4-hydroxy-3-methyl-2-butenyl)-2-(3-methyl-2-butenyl)-3′,4′-didehydro-l′,2′-dihydro-b,y-caroten-1′-ol and bacterioruberin. A particular carotenoid of the embodiments is β-carotene.

The medium to high viscosity vegetable oil or oil mixture is preferably selected from a group consisting of sesame oil, olive oil, almond oil, apricot kernel oil, avocado oil, evening primerose oil, grapeseed oil, hazelnut oil, jojoba oil, pumpkiness oil, roseship oil, safflower oil, walnut oil, wheatgerm oil, sunflower oil and a mixture thereof. In a preferred embodiment, the medium to high viscosity vegetable oil is selected from a group consisting of sesame oil, almond oil, olive oil, sunflower oil and a mixture thereof.

In a particular embodiment, the medium to high viscosity vegetable oil is sesame oil. In such a particular embodiment, the lipophilic component of the composition comprises sesame oil at 40-60% by weight of the lipophilic component and the low viscous triglyceride or the mixture of low viscous triglycerides and/or the low viscous mineral oil or the mixture of low viscous mineral oils at 40-60% by weight of the lipophilic component.

Sesame oil typically comprises about 39.7% by weight of monounsaturated fats and about 41.7% by weight of polyunsaturated fats, i.e. unsaturated fats above 80% by weight. The monounsaturated fats of sesame oil include plamitoleic acid, typically up to 0.5%; oleic acid, typically within 35 to 50%; and eicosenoic acid, typically up to 1%. The polyunsaturated fats of sesame oil include linoleic acid, typically to 50%; and linolenic acid, typically up to 1%. Sesame oil comprises several antioxidants including sesmin, sesamol, sesamolin, x-tocopherol (vitamin E) and vitamin K.

Almond oil typically comprises about 62% monounsatured oleic acid, 29% linoleic acid (a polyunsatured fatty acid) and 9% saturated fatty acid. Almond oil comprises antioxidants including vitamin E.

Olive oil typically comprises more than 85% by weight of unsatured fatty acids, including oleic acid at 70% (monounsatured), palmitoleic acid at 0.3-3.5% (monounsatured), linoleic acid at 15% (polyunsaturated) and α-linolenic acid at 0.5% (polyunsaturated). Saturated fatty acids include palmitic acid at 13% and stearic acid at 1.5%. Olive oil comprises high levels of antioxidants, including polyphenols and vitamin E.

Sunflower oil typically comprises oleic oil (monunsatured) at 30% by weight, linoleic acid (polyunsatured) at 59% by weight, palmitic and stearic acid (saturated) at 5% and 6% by weight, respectively. Sunflower oil comprises antioxidants, including vitamin E.

The lipophilic component comprises, in an embodiment, a low viscous triglyceride or a mixture of low viscous triglycerides in addition to the medium to high viscosity vegetable oil or oil mixture, such as sesame oil. In another embodiment, the lipophilic component comprises a low viscous mineral oil or a mixture of low viscous mineral oils in addition to the medium to high viscosity vegetable oil or oil mixture, such as sesame oil. In a further variant, low viscous triglyceride(s) and low viscous mineral oil(s) are present in the lipophilic component in addition to the medium to high viscosity vegetable oil or oil mixture, such as sesame oil. However, it is typically sufficient to include only one of low viscous triglyceride(s) and low viscous mineral oil(s) in the lipophilic component in order to obtain the desired characteristics of the composition.

The low viscous triglyceride is preferably a triglyceride having a viscosity no higher than 40 cP at room temperature (20° C.), preferably having a viscosity no higher than 35 cP at room temperature (20°).

The low viscous triglyceride is preferably selected among medium-chain triglycerides (MCTs) containing 6-12 carbon fatty acid esters of glycerol.

In a preferred embodiment, the low viscous triglyceride is selected from a group consisting of capric triglyceride and caprylic triglyceride. Correspondingly, the mixture of low viscous triglycerides is preferably a mixture of capric triglyceride and caprylic triglyceride.

Capric triglyceride is a capric acid ester of glycerol. Capric acid, also referred to as decanoic acid, CH₃(CH₂)₈COOH, is a C10 medium-chain fatty acid. Caprylic triglyceride is a caprylic acid ester of glycerol. Caprylic acid, also referred to as octanoic acid, CH₃(CH₂)₆COOH, is a C8 medium-chain fatty acid.

The mixture of capric and caprylic triglycerides is preferably a mixture of capric triglyceride at 20-50% by weight of the mixture and caprylic triglyceride at 50-80% by weight of the mixture. In a particular embodiment, the mixture is preferably a mixture of capric triglyceride at 25-40% by weight of the mixture and caprylic triglyceride at 60-75% by weight of the mixture.

In an embodiment, the lipophilic component of the composition consists of the medium to high viscosity vegetable oil or oil mixture, such as sesame oil, and the low viscous triglyceride or the mixture of low viscous triglycerides. In such a case, the lipophilic component preferably consists of the medium to high viscosity vegetable oil or oil mixture, such as sesame oil, at X % by weight of the lipophilic component and the low viscous triglyceride or the mixture of low viscous triglycerides at 100-X % by weight of the lipophilic component, wherein X ε40-60% by weight of the lipophilic component.

In a preferred embodiment, the lipophilic component consists of the medium to high viscosity vegetable oil or oil mixture, such as sesame oil, at X % by weight of the lipophilic component and a mixture of capric and caprylic triglycerides at 100-X % by weight of the lipophilic component. In an embodiment, X ε40-60% by weight of the lipophilic component, preferably X ε45-55% by weight of the lipophilic component, more preferably X=50% by weight of the lipophilic component.

Thus, preferred variants of liphophilic components include:
the medium to high viscosity vegetable oil or oil mixture, such as sesame oil, at 40% by weight of the lipophilic component and a mixture of capric and caprylic triglycerides at 60% by weight of the lipophilic component;
the medium to high viscosity vegetable oil or oil mixture, such as sesame oil, at 41% by weight of the lipophilic component and a mixture of capric and caprylic triglycerides at 59% by weight of the lipophilic component;
the medium to high viscosity vegetable oil or oil mixture, such as sesame oil, at 42% by weight of the lipophilic component and a mixture of capric and caprylic triglycerides at 58% by weight of the lipophilic component;
the medium to high viscosity vegetable oil or oil mixture, such as sesame oil, at 43% by weight of the lipophilic component and a mixture of capric and caprylic triglycerides at 57% by weight of the lipophilic component;
the medium to high viscosity vegetable oil or oil mixture, such as sesame oil, at 44% by weight of the lipophilic component and a mixture of capric and caprylic triglycerides at 56% by weight of the lipophilic component;
the medium to high viscosity vegetable oil or oil mixture, such as sesame oil, at 45% by weight of the lipophilic component and a mixture of capric and caprylic triglycerides at 55% by weight of the lipophilic component;
the medium to high viscosity vegetable oil or oil mixture, such as sesame oil, at 46% by weight of the lipophilic component and a mixture of capric and caprylic triglycerides at 54% by weight of the lipophilic component;
the medium to high viscosity vegetable oil or oil mixture, such as sesame oil, at 47% by weight of the lipophilic component and a mixture of capric and caprylic triglycerides at 53% by weight of the lipophilic component;
the medium to high viscosity vegetable oil or oil mixture, such as sesame oil, at 48% by weight of the lipophilic component and a mixture of capric and caprylic triglycerides at 52% by weight of the lipophilic component;
the medium to high viscosity vegetable oil or oil mixture, such as sesame oil, at 49% by weight of the lipophilic component and a mixture of capric and caprylic triglycerides at 51% by weight of the lipophilic component; and
the medium to high viscosity vegetable oil or oil mixture, such as sesame oil, at 50% by weight of the lipophilic component and a mixture of capric and caprylic triglycerides at 50% by weight of the lipophilic component.

The low viscous mineral is preferably a mineral oil having a viscosity no higher than 40 cP at room temperature (20° C.), preferably having a viscosity no higher than 35 cP at room temperature (20°).

In an embodiment, the low mineral oil is liquid paraffin, liquid petroleum or variants or mixtures thereof.

A currently preferred example of liquid paraffin is so-called medicinal liquid paraffin, also known as paraffinum liquidum. Medicinal liquid paraffin is a very highly refined mineral oil traditionally used in cosmetics and for medical purposes. Also variants of medicinal liquid paraffin having slightly different alkane blends and that are pharmaceutically acceptable could be used as low viscous mineral oil according to the embodiments.

Non-limiting examples of liquid petroleum that can be used according to the embodiments include petroleum jelly, also referred to as petrolatum.

The surfactant, if present, is preferably a non-ionic surfactant or a mixture of non-ionic surfactants. However, also ionic surfactants, including zwitterionic surfactants, formulated to be non-irritating could be used according to the embodiments.

The surfactant, preferably non-ionic surfactant, is preferably selected from a group consisting of a glycerol monooleate, a sorbitan laurate or a mixture thereof.

Non-irritating as used herein is preferably measured by ISO 10993-10: 2002 Standard, “Biological Evaluation of Medical Devices, Part 10-Test for Irritation and Sensitization”, pp. 6-10, 21.

The propellant, if present, could be selected among currently used propellants for nasal sprays and aerosols. Non-limiting examples include hydrofluoralkane (HFA), such as HFA 134a (1,1,1,2,-tetrafluoroethane) or HFA 227 (1,1,1,2,3,3,3-heptafluoropropane); mixtures of volatile hydrocarbons, such as propane, n-butane and isobutene; dimethyl ether (DME); methyl ethyl ether; chlorofluorocarbons (CFC), or compressed gases, such as nitrogen, carbon dioxide or air.

The propellant or the mixture of propellants is typically optional in the composition. Hence, the composition could be delivered by non-propellant solutions, including manual pump sprays.

The composition of the embodiments is preferably an intranasal composition formulated for intranasal administration.

Preferred uses of the composition of the embodiments other than intranasal administration include administration to the mouth and/or throat, topical administration to the skin, vaginal administration, anal administration or mucosal administration to various mucous membranes.

The composition of the embodiments may optionally comprise a pharmaceutically active agent or a mixture of pharmaceutically active agents. Any such pharmaceutically active agent preferably suitable for intranasal administration can be used according to the embodiments. The at least one pharmaceutically active agent could be intended for local administration in the nose. Alternatively, or in addition, the pharmaceutically active agent could be intended for systemic administration by being taken up by the mucous membrane in the nose and entering the systemic circulation of the patient.

Non-limiting examples of pharmaceutically acceptable agents that can be used according to the embodiments include anti-inflammatory agents, anti-allergenic agents, anti-viral agents, anti-bacterial agents, anti-irritant agents, constricting agents, decongestant agents, anesthetic agents, analgesic agents or mixtures thereof.

Examples of anti-inflammatory agents include non-steroidal anti-inflammatory drugs (NSAIDs) including salicylates, such as aspirin, diflunisal, salsalate, choline magnesium trisalicylate; propionic acid derivates, such ibuprofen, dexibuprofen, naproxen, fenoprofen, ketoprofen, dexketoprofen, flurbiprofen, oxaprozin, loxoprofen; acetic acid derivatives, such as indomethacin, tolmetin, sulindac, etodolac, ketorolac, diclofenac, acelofenac, nabumetone; enolic acid derivatives, such as piroxicam, meloxicam, tenoxicam, droxicam, lornoxicam, isoxicam; anthranilic acid derivatives, such as mefenamic acid, meclofenamic acid, flufenamic acid, tolfenamic acid; selective COX-2 inhibitors, such as celecoxib, rofecoxib, valdecoxib, parecoxib, lumiracoxib, etoricoxib, firocoxib; sulfananilides, such as nimesulfide; licofelone; corticosteroids.

Examples of anti-allergenic agents include antihistamines, such as acrivastine, azelastine, bilastine, brompheniramine, buclizine, bromodiphenhydramine, carbinoxamine, cetirizine, chlorpromazine, cimetidine, cyclizine, chlorpheniramine, chlorodiphenhydramine, clemastine, cyproheptadine, desloratadine, dexbrompheniramine, dexchlorpheniramine, dimenhydrinate, dimetindene, diphenhydramine, doxylamine, ebastine, embramine, famotidine, fexofenadine, hydroxyzine, lafutidine, levocetirizine, loratadine, meclizine, mirtazapine, nizatidine, olopatadine, orphenadrine, phenindamine, heniramine, phenyltoloxamine, promethazine, pyrilamine, quetiapine, ranitidine, roxatidine, rupatadine, tripelennamine, triprolidine; glucocorticoids, such as cortisol (hydrocortisone), cortisone, prednisone, prednisolone, methylprednisolone, dexamethasone, betamethasone, triamcinolone, beclometasone, fludrocortisone acetate, deoxycorticosterone acetate, aldosterone; epinephrine (adrenaline); theophylline; cromolyn sodium; anti-leukotrienes, such as montelukast, zafirlukast; anti-cholinergics; mast cell stabilizers.

Examples of anti-viral agents include amantadine; rimantadine; pleconaril; acyclovir; zidovudine; lamivudine; rifampicin; zanamivir and oseltamivir.

Examples of anti-bacterial agents (sometimes also referred to as anti-septic agents) include bactericides, such as Daquin's solution, sodium benzenesulfochloramide, iodopovidone, urea perhydrate solutions, peracetic acid solutions, sorbic acid, benzoic acid, lactic acid, salicylic acid, hexachlorophene, triclosan, dibromol, benzalkonium, chlorhexidine, octenidine solutions, beta-lactam antibiotics, vancomycin, daptomycin, fluoroquinolones, metronidazole, nitrofurantoin, co-trimoxazole, telithromycin, aminoglycosidic antibiotics; and bacteriostatic agents, such as tetracyclines, sulfonamides, spectinomycin, trimethoprim, chloramphenicol, macrolides, lincosamides, clindamycin, ethambutol, nitrofurantoin, novobiocin, tigecycline, oxazolidinone.

Example of anti-irritant agents include allantoin, menthol, Aloe Vera.

Examples of constricting agents include vasoconstrictors, such as amphetamines, antihistamines, caffeine, methylphenidate, mephedrone, oxymetazoline, phenylephrine, propylhexedrine, pseudoephedrine, tetrahydrozoline hydrochloride.

Examples of decongestant agents include ephedrine, levo-methamphetamine, naphazoline, oxymetazoline, phenylephrine, phenylpropanolamine, propylhexedrine, synephrine, tetrahydrozoline, xylometazoline, pseudoephedrine, tramazoline.

Examples of anesthetic agents include local anesthetic agents in particular ester local anesthetics, such as procaine benzocaine, chloroprocaine, cyclomethycaine, dimethocaine/larocaine, piperocaine, propoxycaine, procaine/novocaine, proparacaine, tetracaine/amethocaine; and amide anesthetics, such as lidocaine articaine, bupivacaine, cinchocaine/dibucaine, etidocaine, levobupivacaine, lidocaine/lignocaine, mepivacaine, prilocaine, ropivacaine, trimecaine.

Examples of analgesic agents include paracetamol; NSAIDs; COX-2 inhibitors; opioids, such as morphine, codeine, oxycodone, hydrocodone, dihydromorphine, pethidine, buprenorphine tramadol, tapentadol; flupirtine; tricyclic antidepressants, such as amitriptyline; nefopam; carbamazepine; gabapentin; pregabalin.

Another example of pharmaceutically active agent is eucalyptus and in particular eucalyptus oil that can be used to relieve the symptoms of influenza and colds. Eucalyptus oil has antibacterial effects on pathogenic bacteria in the respiratory tract. Inhaled eucalyptus oil vapor is a decongestant and treatment for bronchitis. Eucalyptus oil can also control airway mucus hypersecretion and asthma via anti-inflammatory cytokine inhibition. Eucalyptus oil has also anti-inflammatory and analgesic qualities.

In a particular embodiment, the pharmaceutically active agent is selected from a group consisting of a corticosteroid, oxymetazoline, phenylephrine, xylometazoline, naphazoline, eucalyptus, menthol, Aloe Vera, acrivastine, azelastin or a mixture thereof.

Non-limiting examples of compositions of the embodiments include a composition based on sunflower oil, preferably comprising sunflower oil at about 40% by weight of the composition, a mixture of capric and caprylic triglycerides at about 60% by weight of the composition and oxymetazoline at about 0.05% by weight of the composition.

Another example composition is based on olive oil and comprises olive oil at about 40% by weight of the composition, a mixture of capric and caprylic triglycerides at about 55% by weight of the composition, glycerol monooleate at about 5% by weight of the composition and corticosteroid at about 0.025% by weight of the composition.

Other variants of these example compositions are possible by replacing the respective pharmaceutically active substance (oxymetazoline or corticosteroid) by another pharmaceutically active agent.

Another aspect of the embodiments relates to a composition comprising a lipophilic component at 90-99.9% by weight of the composition. The composition also comprises a surfactant or a mixture of surfactant at 0.1-5% by weight of the composition. The lipophilic component comprises, in this aspect, a medium to high viscosity vegetable oil or oil mixture at 40-60% by weight of the lipophilic component and a mixture of capric triglyceride and caprylic triglyceride at 40-60% by weight of the lipophilic component. The medium to high viscosity vegetable oil or oil mixture has a viscosity above 50 cP at room temperature (20° C.) and comprises mono- and/or polyunsaturated fats at at least 40% by weight of the medium to high viscosity vegetable oil or oil mixture.

In an embodiment, the medium to high viscosity vegetable oil or oil mixture comprises at least one antioxidant. The at least one antioxidant can be selected among the previously discussed embodiments. The medium to high viscosity vegetable oil or oil mixture, mono- and/or polyunsatured fats and antioxidants and preferred ranges can advantageously be selected among the previously discussed embodiments. An example of a medium to high viscosity vegetable oil or oil mixture that can be used in this aspect is sesame oil.

The composition is preferably an intranasal composition suitable for intranasal administration.

The previously described preferred mixtures of capric and caprylic triglycerides can advantageously be used also in the composition of the present aspect.

The surfactant or mixture of surfactants can be selected among the previously described embodiments.

In an embodiment, the composition comprises the surfactant or the mixture of surfactants at Y % by weight of the composition and the lipophilic component at 100-Y % by weight of the composition. In such an embodiment, Y ε0.1-5% by weight of the composition.

In a particular embodiment, the surfactant or the mixture of surfactants is present in the composition according to this aspect at an amount of 0.1-1% by weight of the composition, preferably at 0.25-0.75% by weight of the composition, such as at about 0.5% by weight of the composition.

In an embodiment of this aspect, the composition may comprise other constituents than the lipophilic component and the surfactant(s), including other constituents than pharmaceutically active agent(s) and propellant(s).

A further aspect of the embodiments relates to a composition according to any previously described aspects or embodiments for use in treating, preventing and/or relieving symptoms of a nasal medical condition in a human subject.

A related aspect defines use of a composition according to any previously described aspects or embodiments for the manufacture of a medicament for treating, preventing and/or relieving symptoms of a nasal medical condition in a human subject.

Another related aspect defines a method of treating, preventing and/or relieving a nasal medical condition in a human subject. The method comprises intranasally administering a composition according to any previously described aspects or embodiments to the human subject.

In this aspects, the composition is preferably an intranasal composition.

Yet another aspect of the embodiments relates to a composition comprising a lipophilic component at 80-100% by weight of the composition for use in treating, preventing and/or relieving symptoms of a nasal medical condition in a human subject. In this aspect, the lipophilic component comprises a medium to high viscosity vegetable oil or oil mixture at 40-60% by weight of the lipophilic component and a mixture of capric triglyceride and caprylic triglyceride at 40-60% by weight of the lipophilic component. The medium to high viscosity vegetable oil or oil mixture has a viscosity above 50 cP at room temperature (20° C.) and comprises mono- and/or polyunsaturated fats at at least 40% by weight of the medium to high viscosity vegetable oil or oil mixture.

A related aspect defines use of a composition comprising a lipophilic component at 80-100% by weight of the composition for the manufacture of a medicament for treating, preventing and/or relieving symptoms of a nasal medical condition in a human subject. In this related aspect, the lipophilic component comprises a medium to high viscosity vegetable oil or oil mixture at 40-60% by weight of the lipophilic component and a mixture of capric triglyceride and caprylic triglyceride at 40-60% by weight of the lipophilic component. The medium to high viscosity vegetable oil or oil mixture has a viscosity above 50 cP at room temperature (20° C.) and comprises mono- and/or polyunsaturated fats at at least 40% by weight of the medium to high viscosity vegetable oil or oil mixture.

In a preferred embodiment of these aspects, the composition comprises the lipophilic component at 90-100% by weigh to the composition.

Another related aspect defines a method of treating, preventing and/or relieving a nasal medical condition in a human subject. The method comprises intranasally administering a composition comprising a lipophilic component at 90-100% by weight of the composition to the human subject. In this another related aspect, the lipophilic component comprises a medium to high viscosity vegetable oil or oil mixture at 40-60% by weight of the lipophilic component and a mixture of capric triglyceride and caprylic triglyceride at 40-60% by weight of the lipophilic component. The medium to high viscosity vegetable oil or oil mixture has a viscosity above 50 cP at room temperature (20° C.) and comprises mono- and/or polyunsaturated fats at at least 40% by weight of the medium to high viscosity vegetable oil or oil mixture.

In these aspects embodiment, the medium to high viscosity vegetable oil or oil mixture optionally comprises at least one antioxidant. The at least one antioxidant can be selected among the previously discussed embodiments.

In these aspects, the medium to high viscosity vegetable oil or oil mixture is preferably sesame oil.

In these aspects, the composition is preferably an intranasal composition.

The nasal medical condition mentioned above is preferably selected from a group consisting of dry nasal mucous membrane, irritated nasal mucous membrane, sinus infection, rhinosinusitis, allergic rhinitis and nasal swelling.

The compositions of the embodiments comprising at least one pharmaceutically acceptable agent may also, or alternatively, be used for treating, preventing and/or relieving symptoms of other medical conditions, including non-nasal medical conditions, depending on the particular pharmaceutically acceptable agent(s) present in the composition. For instance, the composition could be applied for anti-inflammatory use, pruritus relief, pain reduction, hormone management, either locally or systemically, etc.

A further aspect of the embodiments relates to a nasal spray delivery device. The nasal spray delivery device comprises a nozzle configured to be inserted into a nostril of a human subject. The nasal spray delivery device also comprises a composition container in fluid connection with the nozzle. The composition container comprises a composition according to any previously described aspect or embodiment.

The composition can be delivered from the composition container through the nozzle according to various mechanisms. In an embodiment, the nasal spray delivery device operates as a manually operated pump and thereby comprises a pump mechanism actuated by the user to deliver the composition as a spray or aerosol. The composition container, or a separate propellant container of the nasal spray delivery device, may comprise a propellant for efficient delivery of the composition in the form of a spray or aerosol.

Hence, the nasal spray delivery device may be used for administering a composition according to the embodiments to a human subject, such as for the purpose of treating, preventing and/or relieving symptoms of a nasal medical condition.

Yet another aspect of the embodiments relates to use of a low viscous triglyceride or a mixture of low viscous triglycerides and/or a low viscous mineral oil or a mixture of low viscous mineral oils as an additive to a composition comprising a medium to high viscosity vegetable oil or oil mixture to improve spray characteristics during administration of the composition as a spray or aerosol. The medium to high viscosity vegetable oil or oil mixture has a viscosity above 50 cP at room temperature (20° C.) and comprises mono- and/or polyunsaturated fats at at least 40% by weight of the medium to high viscosity vegetable oil or oil mixture.

In an embodiment, the medium to high viscosity vegetable oil or oil mixture comprises at least one antioxidant. The at least one antioxidant can be selected among the previously discussed embodiments.

In an embodiment, the composition comprises the medium to high viscosity vegetable oil at a concentration of 40-60% by weight of the lipophilic component.

In an embodiment, the medium to high viscosity vegetable oil or oil mixture is sesame oil.

In a particular embodiment, improve spray characteristics means improve adsorption onto skin or mucous membrane during administration of the composition as a spray or aerosol.

In another particular embodiment, improve spray characteristics means increase spray area of the spray or aerosol when administered to skin or mucous membrane.

In a further particular embodiment, improve spray characteristics means improve adsorption onto skin or mucous membrane as mentioned above and increase spray area as mentioned above.

### EXPERIMENTS

Properties of nasal sprays of the embodiments were compared to reference nasal sprays based on pure medium to high viscosity vegetable oils using a sample canister in the form of a precompressed spray pump.

### Experiment 1

In the present experiment a first test nasal spray of the embodiments containing, in addition to sesame oil and low viscous triglycerides, a surfactant was tested and compared to a reference nasal spray consisting solely of sesame oil.

The reference nasal spray consisted of 100% by weight of pure sesame oil (winterized refined sesame oil (Pharmacopoea Europaea (Ph Eur)), Textron Tecnica, S. L, Spain). The test nasal spray of the embodiments consisted of 49.75% by weight of pure sesame oil (winterized refined sesame oil (Ph Eur), Textron Tecnica, S. L., Spain), 49.75% by weight of capric and caprylic triglycerides (Radia 7101 (Ph Eur), Vendico Chemical AB, Sweden) and 0.50% by weight of glycerol monooleate (Cithrol™ GMO HP (Ph Eur), CRODA).

### Organoleptic Evaluation

Table 1 below lists the results of organoleptic evaluation of spray delivered products. Briefly, the organoleptic evaluation involved the following procedure:
1) Shake the sample canister for 3-5 s.
2) Spray 1 s for the sample canister to pre-adjust the spray.
3) Keep the sample canister about 15 cm from the arm.
4) Spray 1 s with the sample canister on the inside of the arm. Move the spray gradually down from the elbow to hand, or in the opposite direction.
   a. Note if the spray comes out as a fine mist or as a non-even spray containing many individual droplet of different sizes.
   b. Note the appearance of the spray on the arm. Is it evenly distributed o non-evenly distributed with clumps.
5) Use the hand to gently rub in the sprayed sample on the arm.
   a. Note if the spray feels greasy, non-greasy or watery.
   b. Note if the spray goes in quickly into the skin.
6) Wait 1 minute and feel on the sprayed sample on the arm.
   a. Does it feel sticky or not.
   b. Does it feel smooth and soft, or does it have a rough feeling.

TABLE 1
Organoleptic evaluation of nasal sprays

| | Adsorption | | | Easiness to |
|---|---|---|---|---|
| Nasal spray | onto skin | Feeling | Stickiness | smear out on skin |
| Reference | Difficult | Soft | Non-sticky | Easy |
| Test | Easy | Soft | Non-sticky | Easy |

The test nasal spray is similar to the reference nasal spray but adsorbs significantly better onto the skin. Hence, the addition of low viscous triglycerides does not negatively affect the soft feeling, non-stickiness and easiness to smear out of pure sesame oil. However, the addition of the low viscous triglycerides improved the adsorption onto the skin of the composition.

### Spray Pattern Testing on Paper

Spray pattern testing was conducted according to a spray pattering testing of spray delivered products. Briefly, the spray pattering testing involved:
1) Place the sample canister in 25° C. water bath for 30 minutes.
2) Shake the sample canister for 4 s.
3) Actuate the pump and spray 5 times on a red A4 paper at 5 cm distance from the paper.
4) Measure the diameter (circular spray pattern) or the width and height (elliptical spray pattern).
5) Note if the spray is evenly distributed or if the spray is concentrated to the center alternatively to the edges.

A circular spray pattern form was obtained in the testing. Samples of the test and reference nasal sprays were each actuated once on a paper. The actuation was done at a vertical position spraying onto the paper from below at a 5 cm distance from the paper.

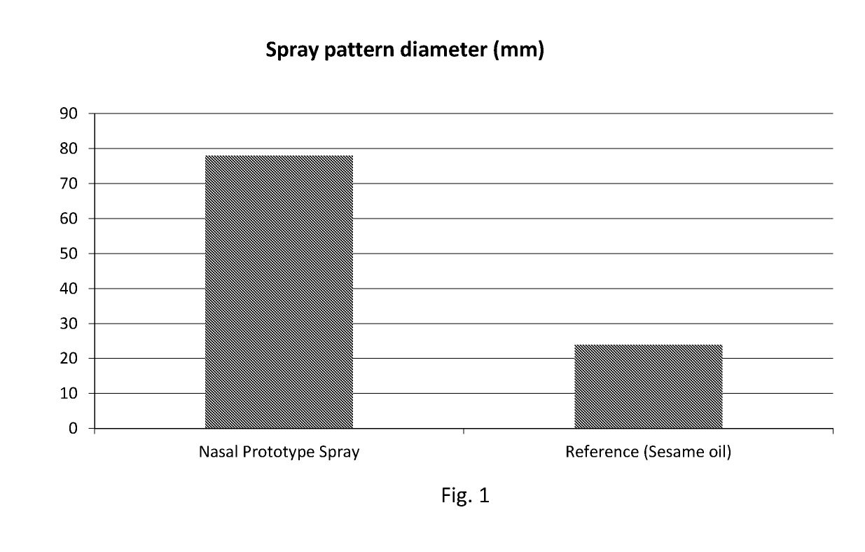

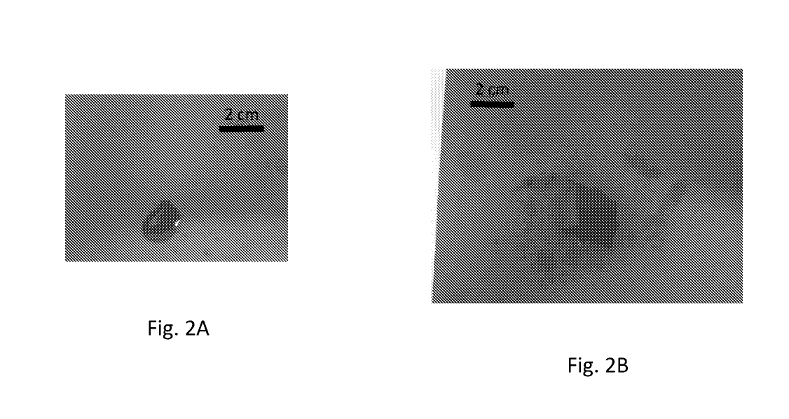

FIG. 1 illustrates the average spray pattern diameter for the test and reference nasal sprays. The test nasal spray achieved a significantly larger average spray pattern diameter (78 mm) as compared to the reference nasal spray (24 mm). FIG. 2 illustrate papers showing the spray patterns of the reference nasal spray (FIG. 2A) and the test nasal spray (FIG. 2B).

### Vertical Flow Testing

A test subject had his arm in a vertical position. A nasal spray was actuated one time, at the top of the arm. The delivered product was followed down the length of the arm and the distance and time was recorded.

TABLE 2
results of vertical flow analysis on inside of arm

| | Amount added | Flow | Flow |
|---|---|---|---|
| Nasal spray | on skin (g) | distance (cm) | rate (cm/s) |
| Reference | 0.08 | 13.5 | 0.45 |
| Reference | 0.08 | 13.9 | 0.46 |
| Reference | 0.09 | 12.5 | 0.42 |
| Reference | 0.07 | 13.5 | 0.45 |
| Test | 0.08 | 7.6 | 0.25 |
| Test | 0.07 | 8.0 | 0.27 |
| Test | 0.08 | 6.7 | 0.22 |
| Test | 0.08 | 7.5 | 0.25 |

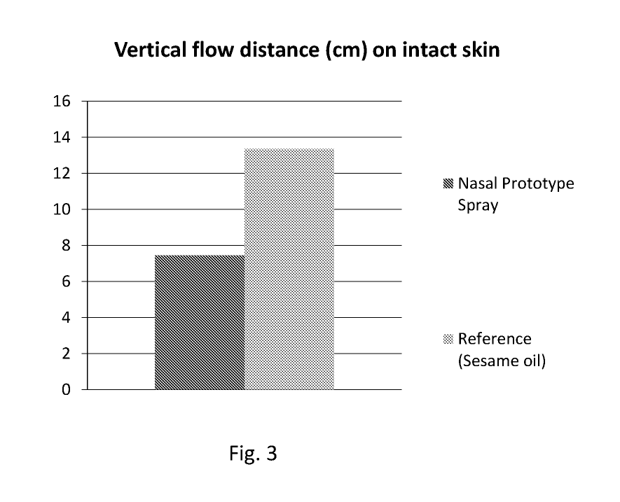

FIG. 3 illustrates a relative comparison of flow rates for the test nasal spray and the reference nasal spray.

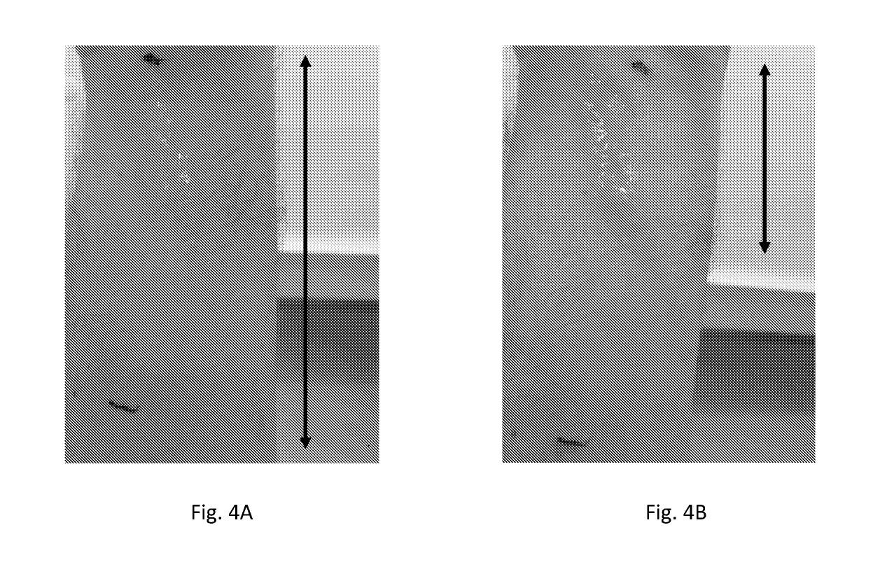

FIG. 4 illustrates vertical flow of reference nasal spray (FIG. 4A) and test nasal spray (FIG. 4B) on intact skin. A test subject had the arm in vertical position. Two labels marked in black indicated a distance of 11 cm. The nasal spray was actuated at the top of the arm and the delivered nasal spray was followed by distance and time down the length of the arm.

### Spray Pattern Testing on Paper Cone

A red paper was cut into circles with 9 cm diameter. Each circle was cut towards the middle and mounted into a cone with the following dimensions: 7.5 cm width and 4.5 cm height. The nasal spray was pre-actuated two times and then placed on a table facing upwards. The cone was placed 1 cm above and the spray was actuated once into the cone.

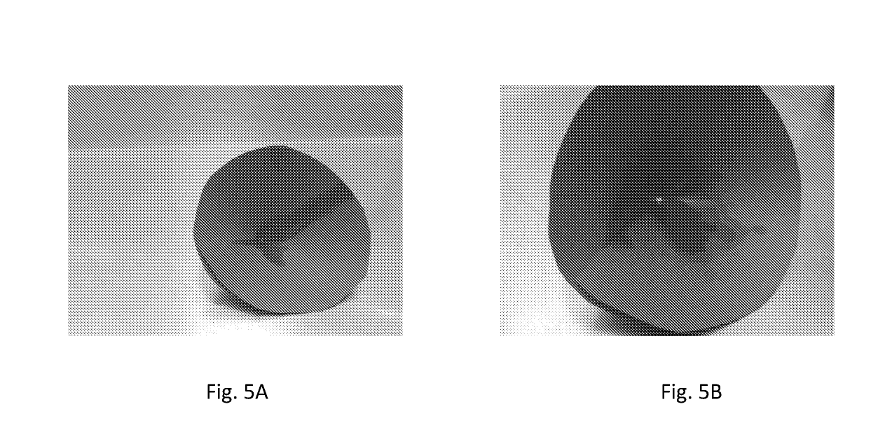

FIG. 5 illustrates the results following actuation of reference nasal spray (FIG. 5A) and test nasal spray (FIG. 5B). In the case of reference nasal spray, the product gave a spray pattern with a central jet of sesame oil resulting in a central oil droplet on the paper. This droplet was thick and was quickly running off the paper. On the contrary, the test nasal spray resulted in a spray mist that targeted the center of the cone in a wider pattern but also with smaller droplets with a non-running product.

### Experiment 2

In the present experiment a second test nasal spray of the embodiments containing sesame oil and low viscous triglycerides was tested and compared to a reference nasal spray consisting solely of sesame oil.

The reference nasal spray consisted of 100% by weight of pure sesame oil (winterized refined sesame oil (Pharmacopoea Europaea (Ph Eur)), Textron Tecnica, S. L, Spain). The test nasal spray of the embodiments consisted of 50% by weight of pure sesame oil (winterized refined sesame oil (Ph Eur), Textron Tecnica, S. L., Spain) and 50% by weight of capric and caprylic triglycerides (Radia 7101 (Ph Eur), Vendico Chemical AB, Sweden). Hence, the test nasal spray lacked any surfactant in this experiment.

### Organoleptic Evaluation

Table 3 below lists the results of organoleptic evaluation of spray delivered products conducted as previously described for Experiment 1.

TABLE 3
Organoleptic evaluation of nasal sprays

| | Adsorption | | | Easiness to |
|---|---|---|---|---|
| Nasal spray | onto skin | Feeling | Stickiness | smear out on skin |
| Reference | Difficult | Soft | Non-sticky | Easy |
| Test | Easy | Soft | Non-sticky | Easy |

The test nasal spray is similar to the reference nasal spray but adsorbs better onto the skin, which is a significant advantage. Hence, the addition of low viscous triglycerides does not negatively affect the soft feeling, non-stickiness and easiness to smear out of pure sesame oil. However, the addition of the low viscous triglycerides improved the adsorption onto the skin of the composition. The same positive results are, thus, obtained for a test nasal spray lacking any surfactant (compare with Table 1).

### Spray Pattern Testing on Paper

Spray pattern testing was conducted according to the spray pattering testing of spray delivered products as previously described for Experiment 1. A circular spray pattern form was obtained for the reference nasal spray and an elliptical spray pattern form was obtained for the test nasal spray in the testing. Samples of the test and reference nasal sprays were each actuated once on a paper. The actuation was done at a vertical position spraying onto the paper from below at a 5 cm distance from the paper.

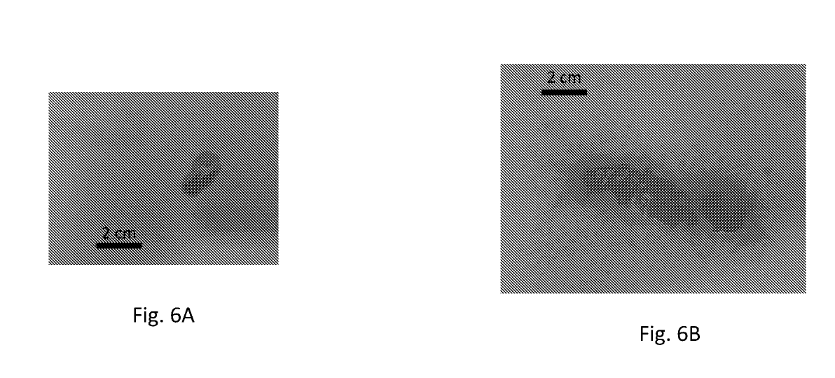

The reference nasal spray resulted in an average spray pattern diameter of 20 mm (n=4). The test nasal spray resulted in an elliptical spray pattern with an average height of 86.25 mm and an average width of 42.50 mm (n=4). The test nasal spray achieved a significantly larger average spray pattern area as compared to the reference nasal spray. FIG. 6 illustrate papers showing the spray patterns of the reference nasal spray (FIG. 6A) and the test nasal spray (FIG. 6B).

### Vertical Flow Testing

A test subject had his arm in a vertical position. A nasal spray was actuated one time, at the top of the arm. The delivered product was followed down the length of the arm and the distance and time was recorded.

TABLE 4
results of vertical flow analysis on inside of arm

| | Amount added | Flow | Flow |
|---|---|---|---|
| Nasal spray | on skin (g) | distance (cm) | rate (cm/s) |
| Reference | 0.08 | 13.0 | 0.43 |
| Reference | 0.08 | 14.2 | 0.47 |
| Reference | 0.08 | 14.0 | 0.47 |
| Reference | 0.08 | 13.2 | 0.44 |
| Test | 0.08 | 7.2 | 0.24 |
| Test | 0.09 | 8.0 | 0.27 |
| Test | 0.09 | 9.0 | 0.30 |
| Test | 0.08 | 8.0 | 0.27 |

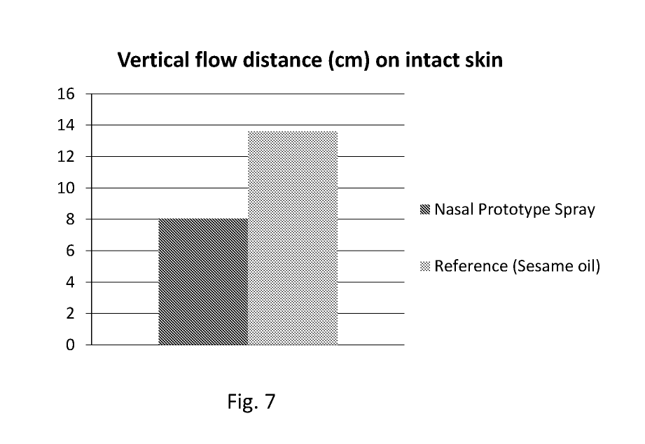

FIG. 7 illustrates a relative comparison of flow rates for the test nasal spray and the reference nasal spray.

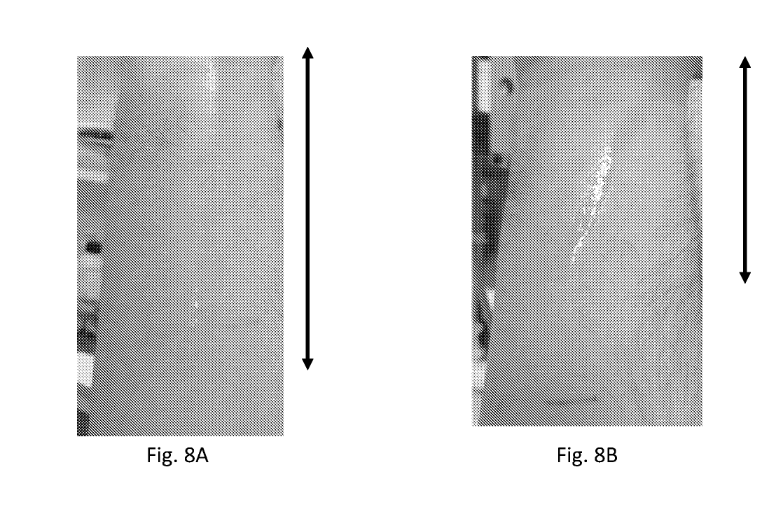

FIG. 8 illustrates vertical flow of reference nasal spray (FIG. 8A) and test nasal spray (FIG. 8B) on intact skin. A test subject had the arm in vertical position. Two labels marked in black indicated a distance of 11 cm. The nasal spray was actuated at the top of the arm and the delivered nasal spray was followed by distance and time down the length of the arm.

### Spray Pattern Testing on Paper Cone

A red paper was cut into circles with 9 cm diameter. Each circle was cut towards the middle and mounted into a cone with the following dimensions: 7.5 cm width and 4.5 cm height. The nasal spray was pre-actuated two times and then placed on a table facing upwards. The cone was placed 1 cm above and the spray was actuated once into the cone.

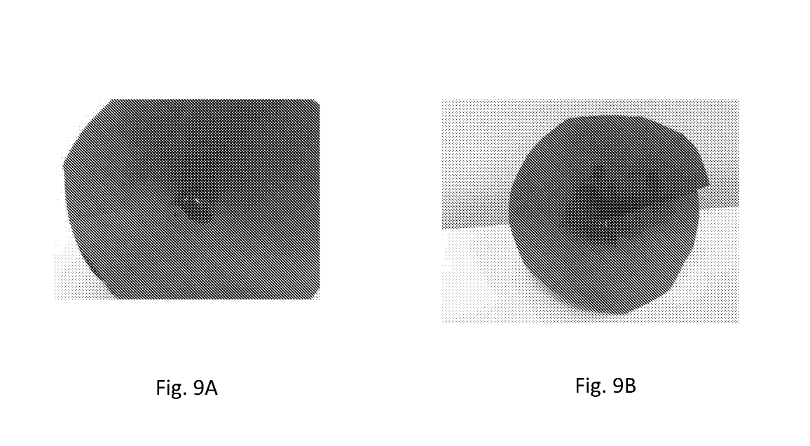

FIG. 9 illustrates the results following actuation of reference nasal spray (FIG. 9A) and test nasal spray (FIG. 9B). In the case of reference nasal spray, the product gave a spray pattern with a central jet of sesame oil resulting in a central oil droplet on the paper. On the contrary, the test nasal spray resulted in a spray mist that targeted the center of the cone in a wider pattern but also with smaller droplets with a less running product.

### Experiment 3

In the present experiment various test nasal sprays of the embodiments containing low viscous triglycerides, an optional surfactant and different medium to high viscosity vegetable oils were tested and compared to respective reference nasal sprays consisting solely of the vegetable oils.

The test nasal sprays of the embodiments consisted of:
1) 49.75% by weight of pure sesame oil (winterized refined sesame oil (Ph Eur), Textron Tecnica, S. L., Spain), 49.75% by weight of capric and caprylic triglycerides (Radia 7101 (Ph Eur), Vendico Chemical AB, Sweden) and 0.50% by weight of glycerol monooleate (Cithrol™ GMO HP (Ph Eur), CRODA);
2) 55% by weight Seatons Almond oil (Croda Europe Limited), 40% by weight capric and caprylic triglycerides (Radia 7101 (Ph Eur), Vendico Chemical AB, Sweden) and 5% by weight of glycerol monooleate (Cithrol™ GMO HP (Ph Eur), CRODA);
3) 40% by weight Seatons Sunflower oil, 60% by weight capric and caprylic triglycerides (Radia 7101 (Ph Eur), Vendico Chemical AB, Sweden); and
4) 40% Super refined Olive oil (Croda Europe Limited), 55% by weight capric and caprylic triglycerides (Radia 7101 (Ph Eur), Vendico Chemical AB, Sweden) and 5% by weight of glycerol monooleate (Cithrol™ GMO HP (Ph Eur), CRODA).

The reference nasal sprays consisted of:
1) 100% by weight of pure sesame oil (winterized refined sesame oil (Pharmacopoea Europaea (Ph Eur)), Textron Tecnica, S. L, Spain);
2) 100% by weight of Seatons Almond oil (Croda Europe Limited);
3) 100% by weight of Seatons Sunflower oil (Croda Europe Limited); and
4) 100% by weight of Super refined Olive oil (Croda Europe Limited).

### Spray Pattern Testing on Paper

Spray pattern testing was conducted according to the spray pattering testing of spray delivered products as previously described for Experiment 1. A circular spray pattern form was obtained for the reference nasal sprays and an elliptical spray pattern form was obtained for the test nasal sprays in the testing. Samples of the test and reference nasal sprays were each actuated once on a paper. The actuation was done at a vertical position spraying onto the paper from below at a 5 cm distance from the paper.

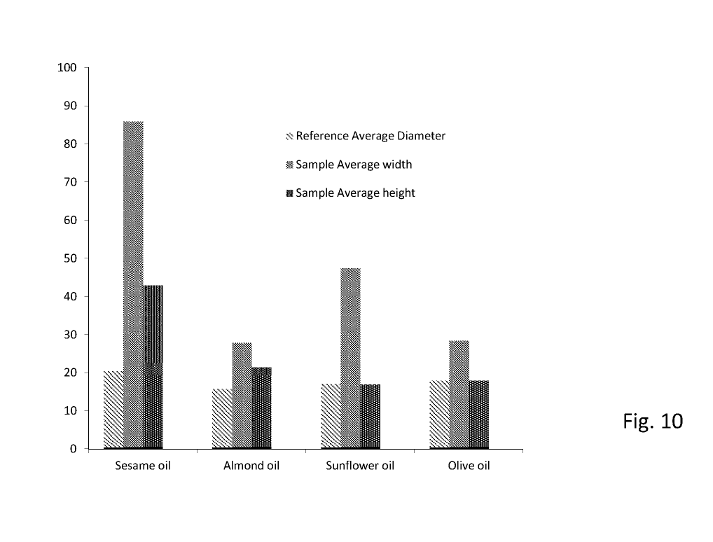

The test nasal sprays achieved significantly larger average spray pattern areas as compared to the reference nasal sprays. FIGS. 6A and 6B illustrate papers showing the spray patterns of the reference sesame oil nasal spray (FIG. 6A) and the test sesame oil nasal spray (FIG. 6B). FIG. 10 illustrates the average diameter (reference samples) and average width and height (test nasal sprays) for all four tested oils (n=4).

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-27. (canceled)

28. A composition consisting of:
a lipophilic component comprising:
a medium to high viscosity vegetable oil or oil mixture at 40-60% by weight of said lipophilic component, said medium to high viscosity vegetable oil or oil mixture having a viscosity above 50 centipoise (cP) at room temperature (20° C.) and comprising mono- and/or polyunsaturated fats at at least 40% by weight of said medium to high viscosity vegetable oil or oil mixture; and
a low viscous triglyceride or a mixture of low viscous triglycerides and/or a low viscous mineral oil or a mixture of low viscous mineral oils at 40-60% by weight of said lipophilic component;
a surfactant or a mixture of surfactants at 0-5% by weight of said composition;
a pharmaceutically active agent or a mixture of pharmaceutically active agents at 0-5% by weight of said composition; and
a propellant or a mixture of propellants at 0-20% by weight of said composition.

29. The composition according to claim 28, wherein said medium to high viscosity oil or oil mixture comprises at least one antioxidant.

30. The composition according to claim 29, wherein said at least one antioxidant is selected from a group consisting of vitamin E, vitamin K and carotenoids.

31. The composition according to claim 28, wherein said medium to high viscosity vegetable oil or oil mixture has a viscosity within an interval of 50 to 100 cP at room temperature (20° C.)

32. The composition according to claim 31, wherein said medium to high viscosity vegetable oil or oil mixture has a viscosity within an interval of 60 to 90 cP at room temperature (20° C.).

33. The composition according to claim 28, wherein said mono- and/or polyunsaturated fats are selected from a group consisting of palmitoleic acid, oleic acid, linoleic acid, linolenic acid, eicosenoic acid and erucic acid.

34. The composition according to claim 28, wherein said medium to high viscosity vegetable oil or oil mixture comprises said mono- and/or polyunsaturated fats at at least 50% by weight, preferably at at least 60% by weight, of said medium to high viscosity vegetable oil or oil mixture.

35. The composition according to claim 28, wherein said medium to high viscosity vegetable oil or oil mixture is selected from a group consisting of sesame oil, olive oil, almond oil, apricot kernel oil, avocado oil, evening primerose oil, grapeseed oil, hazelnut oil, jojoba oil, pumpkiness oil, roseship oil, safflower oil, walnut oil, wheatgerm oil, sunflower oil and a mixture thereof.

36. The composition according to claim 35, wherein said medium to high viscosity vegetable oil is sesame oil.

37. The composition according to claim 28, wherein said composition is an intranasal composition formulated for intranasal administration.

38. The composition according to claim 28, wherein said low viscous mineral oil is a liquid paraffin or liquid petroleum.

39. The composition according to claim 28, wherein said lipophilic component consists of:
said medium to high viscosity vegetable oil or oil mixture at X % by weight of said lipophilic component, wherein X ε40-60% by weight of said lipophilic component; and
said low viscous triglyceride or said mixture of low viscous triglycerides at 100-X % by weight of said lipophilic component.

40. The composition according to claim 28, wherein said low viscous triglyceride is a triglyceride having a viscosity no higher than 40 cP at room temperature (20° C.).

41. The composition according to claim 40, wherein said low viscous triglyceride is a triglyceride having a viscosity no higher than 35 cP at room temperature.

42. The composition according to claim 28, wherein said low viscous triglyceride or said mixture of low viscous triglyceride is selected from a group consisting of capric triglyceride, caprylic triglyceride and a mixture of capric triglyceride and caprylic triglyceride.

43. The composition according to claim 42, wherein said mixture of low viscous triglyceride is a mixture of capric triglyceride at 20-50% by weight of said mixture and caprylic triglyceride at 50-80% by weight of said mixture.

44. The composition according to claim 43, wherein said mixture of low viscous triglyceride is a mixture of capric triglyceride at 25-40% by weight of said mixture and caprylic triglyceride at 60-75% by weight of said mixture.

45. The composition according to claim 28, wherein said composition consists of:
said lipophilic component at 90-100% by weight of said composition;
said surfactant or said mixture of surfactants at 0-5% by weight of said composition;
said pharmaceutically active agent or said mixture of pharmaceutically active agents at 0-5% by weight of said composition; and
said propellant or said mixture of propellants at 0-10% by weight of said composition.

46. The composition according to claim 45, wherein said pharmaceutically active agent or said mixture of pharmaceutically active agents is selected from a group consisting of an anti-inflammatory agent, an anti-allergenic agent, an anti-viral agent, an anti-bacterial agent, an anti-irritant agent, a constricting agent, a decongestant agent, an anesthetic agent, an analgesic agent or a mixture thereof.

47. The composition according to claim 46, wherein said pharmaceutically active agent or said mixture of pharmaceutically active agents is selected from a group consisting of a corticosteroid, oxymetazoline, phenylephrine, xylometazoline, naphazoline, eucalyptus, menthol, Aloe Vera, acrivastine, azelastin or a mixture thereof.

48. The composition according to claim 28, wherein said surfactant or said mixture of surfactants is a non-ionic surfactant or a mixture of non-ionic surfactants.

49. The composition according to claim 21, wherein said non-ionic surfactant or said mixture of non-ionic surfactants is selected from a group consisting of a glycerol monooleate, sorbitan laurate, or a mixture thereof.

50. A composition comprising:
a lipophilic component at 90-99.9% by weight of said composition, said lipophilic component comprises:
a medium to high viscosity vegetable oil or oil mixture at 40-60% by weight of said lipophilic component, said medium to high viscosity vegetable oil or oil mixture having a viscosity above 50 centipoise (cP) at room temperature (20° C.) and comprising mono- and/or polyunsaturated fats at at least 40% by weight of said medium to high viscosity vegetable oil or oil mixture; and
a mixture of capric triglyceride and caprylic triglyceride at 40-60% by weight of said lipophilic component; and
a surfactant or a mixture of surfactants at 0.1-5% by weight of said composition.

51. The composition according to claim 50, wherein said medium to high viscosity vegetable oil is sesame oil.

52. A method of treating, preventing and/or relieving a nasal medical condition selected from a group consisting of dry nasal mucous membrane, irritated nasal mucous membrane, sinus infection, rhinosinusitis, allergic rhinitis, and nasal swelling in a human subject, said method comprises intranasally administering a composition according to claim 28 to said human subject.

53. A method of treating, preventing and/or relieving a nasal medical condition selected from a group consisting of dry nasal mucous membrane, irritated nasal mucous membrane, sinus infection, rhinosinusitis, allergic rhinitis, and nasal swelling in a human subject, said method comprises intranasally administering a composition according to claim 50 to said human subject.

54. A method of treating, preventing and/or relieving a nasal medical condition selected from a group consisting of dry nasal mucous membrane, irritated nasal mucous membrane, sinus infection, rhinosinusitis, allergic rhinitis, and nasal swelling in a human subject, said method comprises intranasally administering a composition comprising a lipophilic component at 80-100% by weight of said composition to said human subject, preferably comprising said lipophilic component at 90-100% by weight of said composition, said lipophilic component comprises:
a medium to high viscosity vegetable oil or oil mixture at 40-60% by weight of said lipophilic component, said medium to high viscosity vegetable oil or oil mixture having a viscosity above 50 centipoise (cP) at room temperature (20° C.) and comprising mono- and/or polyunsaturated fats at at least 40% by weight of said medium to high viscosity vegetable oil or oil mixture; and
a mixture of capric triglyceride and caprylic triglyceride at 40-60% by weight of said lipophilic component.

55. A nasal spray delivery device comprising:
a nozzle configured to be inserted into a nostril of a human subject; and
a composition container in fluid connection with said nozzle and comprising a composition according to claim 28.

56. A nasal spray delivery device comprising:
a nozzle configured to be inserted into a nostril of a human subject; and
a composition container in fluid connection with said nozzle and comprising a composition according to claim 50.
